(19) United States
(12) Patent Application Publication
(10) Pub. No.: US 2023/0405237 A1
(43) Pub. Date: Dec. 21, 2023

(54) Drug Delivery System, and Barrier Apparatus and Pull Tab Therefor

(71) Applicant: Becton, Dickinson and Company, Franklin Lakes, NJ (US)

(72) Inventors: Steven Zebrowski, Oxford, NJ (US); Eric Clasen, Hillsborough, NJ (US)

(21) Appl. No.: 18/033,858

(22) PCT Filed: Oct. 26, 2021
(86) PCT No.: PCT/US21/56578

§ 371 Date: Apr. 26, 2023

Related U.S. Application Data
(60) Provisional application No. 63/106,204, filed on Oct. 27, 2020.

(51) Int. Cl. A61M 5/32

(52) U.S. Cl. CPC A61M 5/3204

(57) ABSTRACT

A drug delivery system is for injecting a medicament. The drug delivery system includes a housing, a needle having a sharp, and a barrier apparatus. The sharp is configured to move into and out of the housing. The barrier apparatus has a barrier configured to protectively surround at least the sharp of the needle, and a pull tab. The pull tab includes a connecting portion connected to the barrier, a gripping portion located apart from the connecting portion and at least partially external with respect to the housing, and a body portion extending between the connecting portion and the gripping portion. The body portion has a flexible portion for reducing strain on the barrier.

### CROSS-REFERENCE TO RELATED APPLICATION

The present application claims priority to U.S. Provisional Application Ser. No. 63/106,204, filed Oct. 27, 2020, entitled “Drug Delivery System, and Barrier Apparatus and Pull Tab Therefor”, the entire disclosure of which is hereby incorporated by reference in its entirety.

### BACKGROUND

### Field of the Disclosure

The present disclosure relates generally to a drug delivery system for delivering a fluid into the body of a patient by injection. The present disclosure also relates to a barrier apparatus for a drug delivery system. The present disclosure further relates to a pull tab for a barrier apparatus.

### Description of the Related Art

Various types of automatic injection devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. When the volume of fluid or drug to be administered is generally below a certain volume, such as 1 mL, an auto-injector is typically used, which typically has an injection time of about 10 to 15 seconds. When the volume of fluid or drug to be administered is above 1 mL, the injection time generally becomes longer resulting in difficulties for the patient to maintain contact between the device and the target area of the patient's skin. Further, as the volume of drug to be administered becomes larger, increasing the time period for injection becomes desirable. The traditional method for a drug to be injected slowly into a patient is to initiate an IV and inject the drug into the patient's body slowly. Such a procedure is typically performed in a hospital or outpatient setting.

Certain devices allow for self-injection in a home setting and are capable of gradually injecting a liquid therapeutic preparation into the skin of a patient. In some cases, these devices are small enough (both in height and in overall size) to allow them to be “worn” by a patient while the liquid therapeutic preparation is being infused into the patient. These devices typically include a pump or other type of discharge mechanism to force the liquid therapeutic preparation to flow out of a reservoir and into the injection needle. Such devices also typically include a valve or flow control mechanism to cause the liquid therapeutic preparation to begin to flow at the proper time and a triggering mechanism to initiate the injection.

### SUMMARY

In one aspect, a drug delivery system for injecting a medicament is provided. The drug delivery system has a housing, a needle having a sharp, and a barrier apparatus. The sharp is configured to move into and out of the housing. The barrier apparatus includes a barrier configured to protectively surround at least the sharp of the needle, and a pull tab. The pull tab has a connecting portion connected to the barrier, a gripping portion located apart from the connecting portion and at least partially external with respect to the housing, and a body portion extending between the connecting portion and the gripping portion. The body portion has a flexible portion for reducing strain on the barrier.

In another aspect, a barrier apparatus for the aforementioned drug delivery system is provided. In another aspect, a pull tab for the aforementioned barrier apparatus is provided.

In a further aspect, the flexible portion is more flexible than the connecting portion and the gripping portion. The flexible portion may extend from at or about the connecting portion to at or about the gripping portion. Optionally, the flexible portion is a segmented tube. In a further configuration, the flexible portion is a webbed portion. In another configuration, the flexible portion is a coiled portion. The flexible portion may be a stretchable straight segment.

In accordance with a further aspect of the present invention, the gripping portion may have a base having a first diameter, and the connecting portion may have a second diameter less than the first diameter. The gripping portion may also include a protrusion and a frusto-conical shaped portion extending between the base and the protrusion.

In other configurations, the body portion may have a third diameter which is less than the first diameter and the second diameter. The system may further include a removal component, with the gripping portion attached to the removal component. A barrier of the device may include a cylindrical-shaped surface and an internal ledge extending radially inwardly from the cylindrical-shaped surface, and the needle may extend through the internal ledge, with the internal ledge engaging the needle.

The barrier may include an elongated portion and a generally planar portion extending from the elongated portion of the barrier and be disposed generally perpendicular with respect to a longitudinal axis of the needle. The system may further include a removal component having a serrated portion, with the gripping portion attached to the removal component by the serrated portion. In certain configurations, the barrier may include an elastomeric sleeve for maintaining the sterility of the needle.

In accordance with another aspect of the present invention, a barrier apparatus for a drug delivery system includes a housing and a needle having a sharp, the sharp being configured to move into and out of the housing. The barrier apparatus includes a barrier configured to protectively surround at least the sharp of the needle, and a pull tab. The pull tab includes a connecting portion connected to the barrier, a gripping portion disposed apart from the connecting portion and configured to be disposed at least partially external with respect to the housing, and a body portion extending between the connecting portion and the gripping portion. The body portion has a flexible portion for reducing strain on the barrier.

In accordance with certain aspects, the barrier and the pull tab are a unitary component made from one single piece of material. The connecting portion may be connected to the barrier by a press-fit mechanism. The barrier may be substantially enclosed by the connecting portion of the pull tab.

In accordance with yet another aspect of the invention, a pull tab for a barrier apparatus of a drug delivery system includes a connecting portion configured to be connected to the barrier, a gripping portion disposed apart from the connecting portion, and a body portion extending between the connecting portion and the gripping portion. The body portion has a flexible portion for reducing strain on the barrier.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery system, shown without a barrier apparatus and pull tab therefor, according to one aspect of the present concept.

FIG. 2 is a perspective, cross-sectional view of the drug delivery system of FIG. 1 according to one aspect of the present concept.

FIG. 3 is a front, cross-sectional view of the drug delivery system of FIG. 1 according to one aspect of the present concept.

FIG. 4 is a top view of the drug delivery system of FIG. 1 according to one aspect of the present concept, showing a top portion of the housing removed and the drug delivery system in a pre-use position.

FIG. 5 is a top, cross-sectional view of the drug delivery system of FIG. 1 according to one aspect of the present concept, showing the drug delivery system in a pre-use position.

FIG. 6 is a front, cross-sectional view of the drug delivery system of FIG. 1 according to one aspect of the present concept, showing the drug delivery system in a pre-use position.

FIG. 7 is a top view of the drug delivery system of FIG. 1 according to one aspect of the present concept, showing a top portion of the housing removed and the drug delivery system in an initial actuation position.

FIG. 8 is a top, cross-sectional view of the drug delivery system of FIG. 1 according to one aspect of the present concept, showing the drug delivery system in an initial actuation position.

FIG. 9 is a front, cross-sectional view of the drug delivery system of FIG. 1 according to one aspect of the present concept, showing the drug delivery system in an initial actuation position.

FIG. 10 is a top view of the drug delivery system of FIG. 1 according to one aspect of the present concept, showing a top portion of the housing removed and the drug delivery system in a use position.

FIG. 11 is a top, cross-sectional view of the drug delivery system of FIG. 1 according to one aspect of the present concept, showing the drug delivery system in a use position.

FIG. 12 is a front, cross-sectional view of the drug delivery system of FIG. 1 according to one aspect of the present concept, showing the drug delivery system in a use position.

FIG. 13 is a top view of the drug delivery system of FIG. 1 according to one aspect of the present concept, showing a top portion of the housing removed and the drug delivery system in a post-use position.

FIG. 14 is a top, cross-sectional view of the drug delivery system of FIG. 1 according to one aspect of the present concept, showing the drug delivery system in a post-use position.

FIG. 15A is a front, cross-sectional view of the drug delivery system of FIG. 1 according to one aspect of the present concept, showing the drug delivery system in a post-use position.

FIG. 15B is a front, cross-sectional view of the drug delivery system of FIG. 1 according to one aspect of the present concept, showing a pad with the drug delivery system in a pre-use position.

FIG. 15C is a perspective, cross-sectional view of the drug delivery system of FIG. 1 according to one aspect of the present concept, showing a pad with the drug delivery system in a pre-use position.

FIG. 15D is a perspective, cross-sectional view of the drug delivery system of FIG. 1 according to one aspect of the present concept, showing a pad with the drug delivery system in a pre-use position.

FIG. 16 is a partial cross-sectional view of the drug delivery system of FIG. 1 according to one aspect of the present concept, showing a valve assembly.

FIG. 17 is a partial cross-sectional view of the drug delivery system of FIG. 3, showing a barrier apparatus in accordance with a non-limiting embodiment of the disclosed concept.

FIG. 18 is an isometric view of the barrier apparatus of FIG. 17.

FIG. 19 is a partial front view of a portion of the barrier apparatus of FIG. 18.

FIG. 20 is a partial front view of a portion of a barrier apparatus in accordance with an alternative non-limiting embodiment of the disclosed concept.

FIG. 21 is a partial front view of a portion of a barrier apparatus in accordance with an alternative non-limiting embodiment of the disclosed concept.

FIG. 22 is a partial front view of a portion of a barrier apparatus in accordance with an alternative non-limiting embodiment of the disclosed concept.

FIG. 23 is a partial front view of a housing and barrier apparatus in accordance with another non-limiting embodiment of the disclosed concept.

FIG. 24 is a partial front view of a barrier apparatus and in accordance with another non-limiting embodiment of the disclosed concept.

FIG. 25 is a partial front view of a barrier apparatus and needle in accordance with another non-limiting embodiment of the disclosed concept.

FIG. 26 is a partial front view of the drug delivery system of FIG. 1, showing movement of a barrier apparatus in accordance with a non-limiting embodiment of the disclosed concept.

FIG. 27 is a partial front view of the drug delivery system of FIG. 1, showing removal of a barrier apparatus in accordance with a non-limiting embodiment of the disclosed concept.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the concept. Various modifications, equivalents, variations, and alternatives, however, will remain readily apparent to those skilled in the art. Any and all such modifications, variations, equivalents, and alternatives are intended to fall within the spirit and scope of the present concept.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

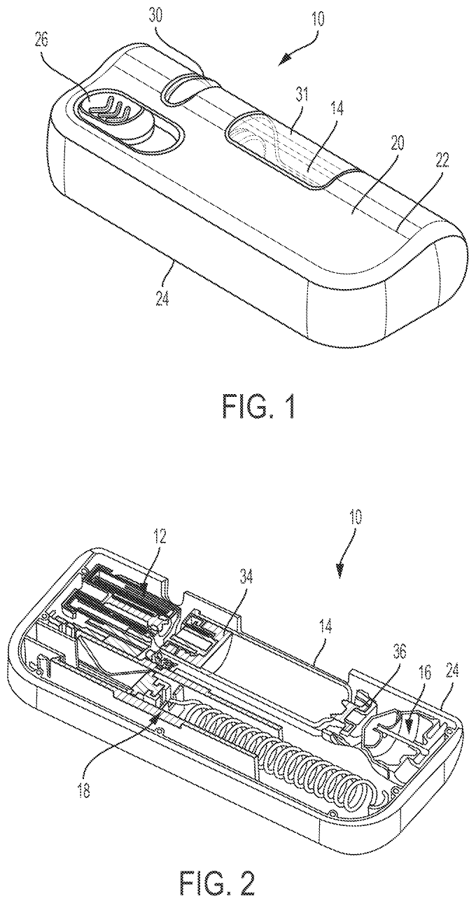

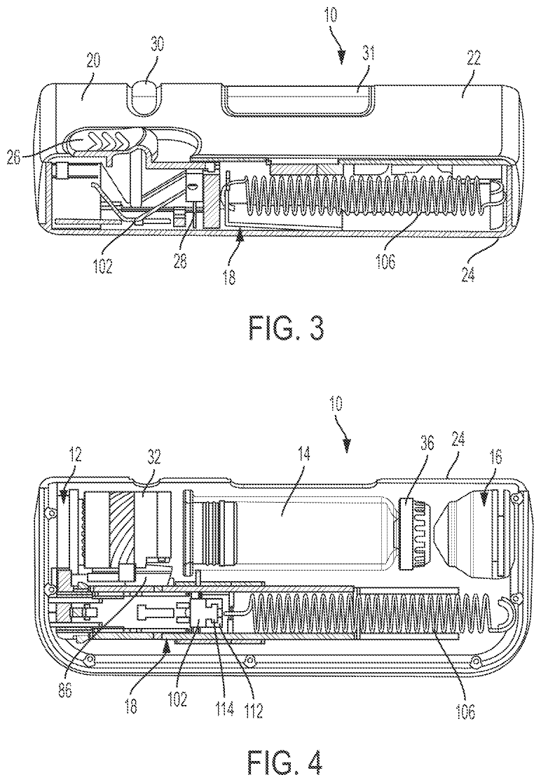

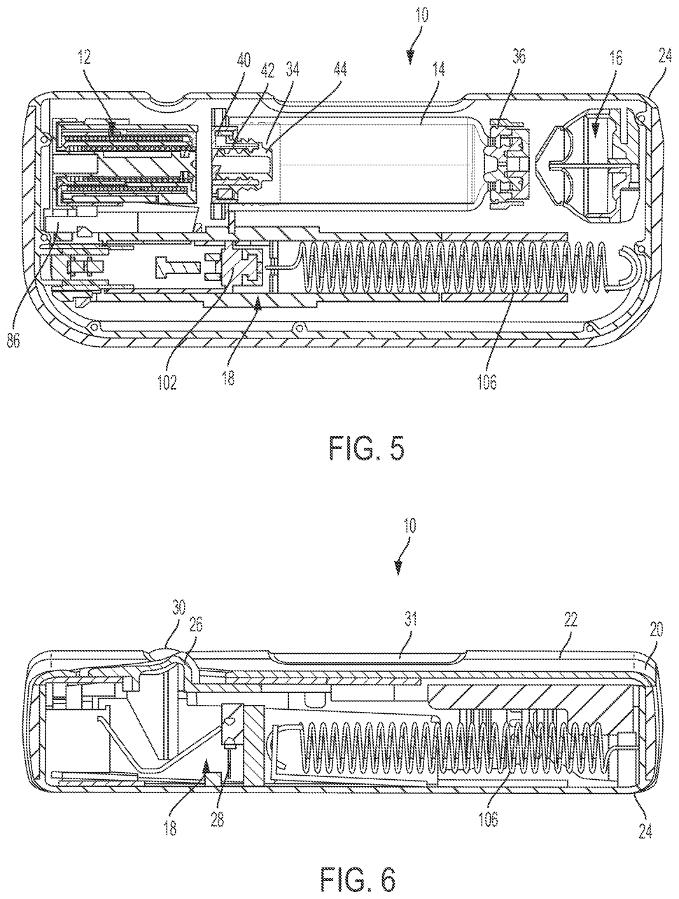

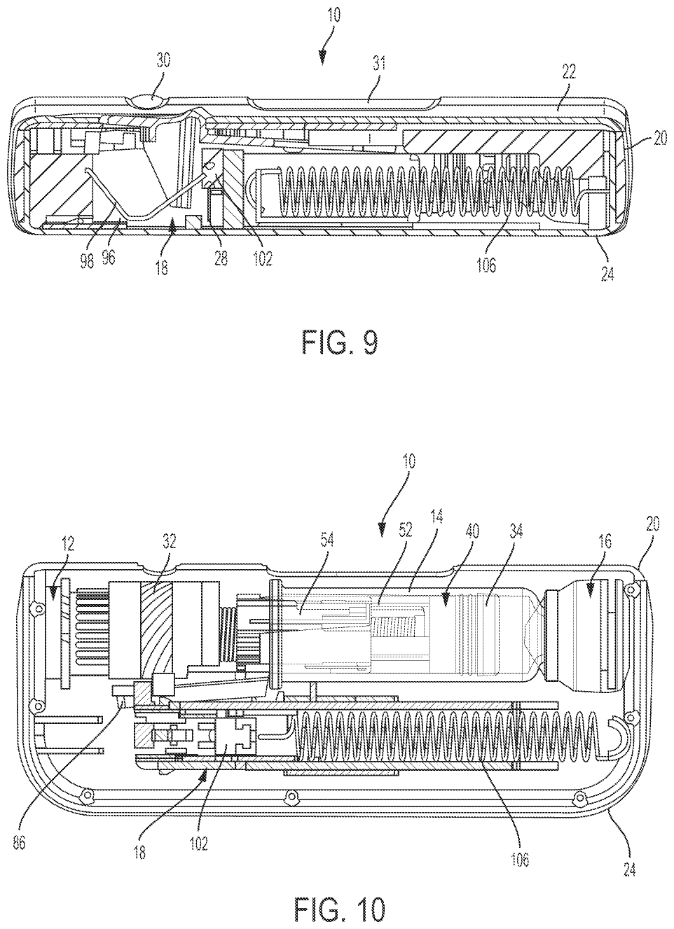

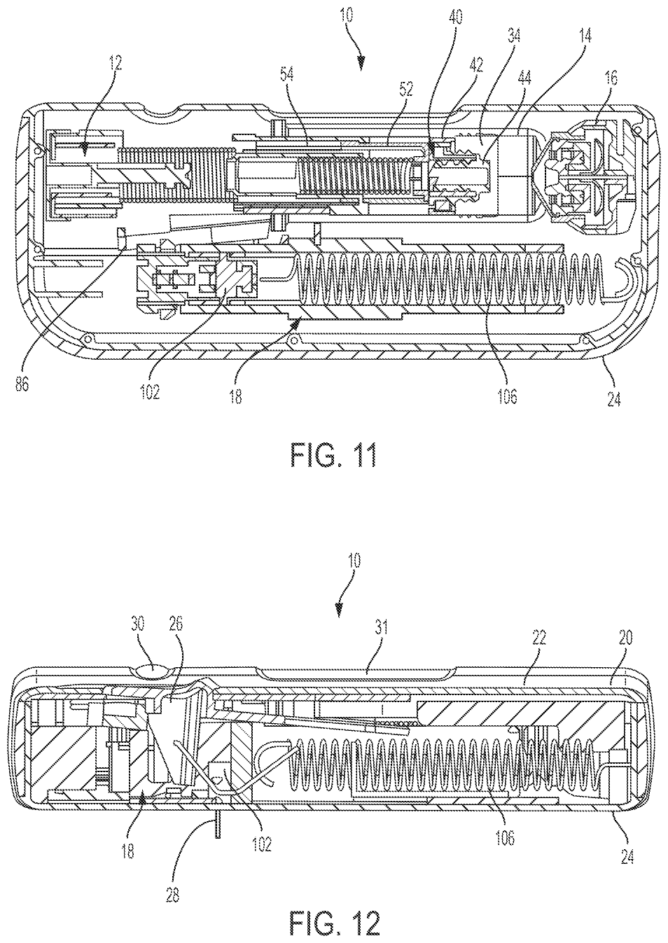

Referring to FIGS. 1-16, a drug delivery system 10 according to one aspect of the present concept includes a drive assembly 12, a container 14, a valve assembly 16, and a needle actuator assembly 18. The drive assembly 12, the container 14, the valve assembly 16, and the needle actuator assembly 18 are at least partially positioned within a housing 20. The housing 20 includes a top portion 22 and a bottom portion 24, although other suitable arrangements for the housing 20 may be utilized. In one aspect, the drug delivery system 10 is an injector device configured to be worn or secured to a user and to deliver a predetermined dose of a medicament provided within the container 14 via injection into the user. The system 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The system 10 may deliver the medicament at a fixed pressure to the user with the rate being variable. The general operation of the system 10 is described below in reference to FIGS. 1-16.

Referring again to FIGS. 1-16, the system 10 is configured to operate through the engagement of an actuation button 26 by a user, which results in a needle 28 of the needle assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a drug delivery system is shown and described in International Publication Nos. 2013/155153 and 2014/179774, which are hereby incorporated by reference in their entirety. The housing 20 of the system 10 includes an indicator window 30 for viewing an indicator arrangement 32 configured to provide an indication to a user on the status of the system 10 and a container window 31 for viewing the container 14. The indicator window 30 may be a magnifying lens for providing a clear view of the indicator arrangement 32. The indicator arrangement 32 moves along with the needle actuator assembly 18 during use of the system 10 to indicate a pre-use status, use status, and post-use status of the system 10. The indicator arrangement 32 provides visual indicia regarding the status, although other suitable indicia, such an auditory or tactile, may be provided as an alternative or additional indicia.

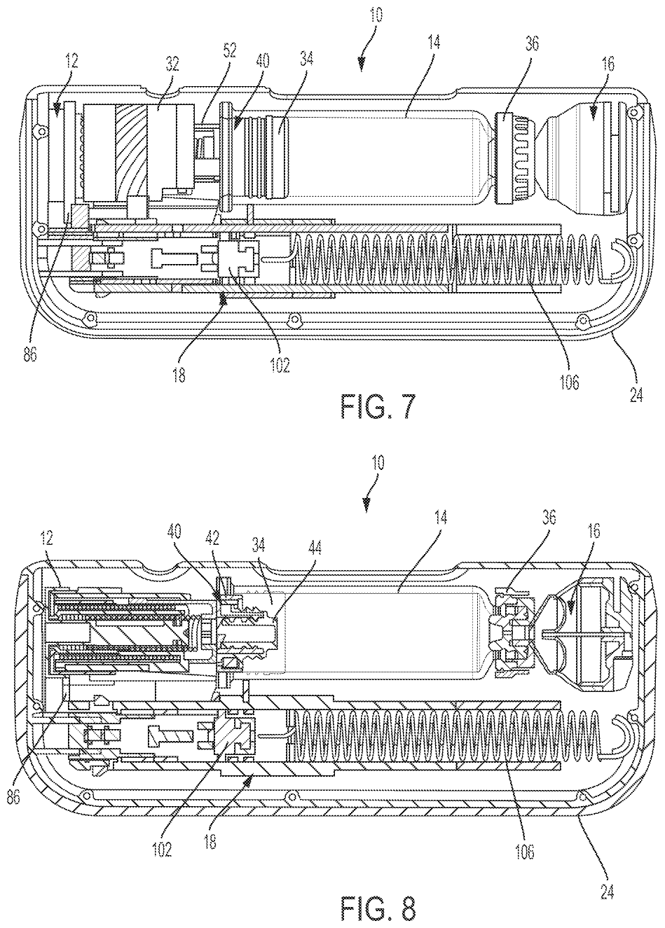

Referring to FIGS. 4-6, during a pre-use position of the system 10, the container 14 is spaced from the drive assembly 12 and the valve assembly 16 and the needle 28 is in a retracted position. During the initial actuation of the system 10, as shown in FIGS. 7-9, the drive assembly 12 engages the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 is configured to engage a stopper 34 of the container 14, which will initially move the entire container 14 into engagement with the valve assembly 16 due to the incompressibility of the fluid or medicament within the container 14. The initial actuation of the system 10 is caused by engagement of the actuation button 26 by a user, which releases the needle actuator assembly 18 and the drive assembly 12 as discussed below in more detail. During the initial actuation, the needle 28 is still in the retracted position and about to move to the extended position to inject the user of the system 10.

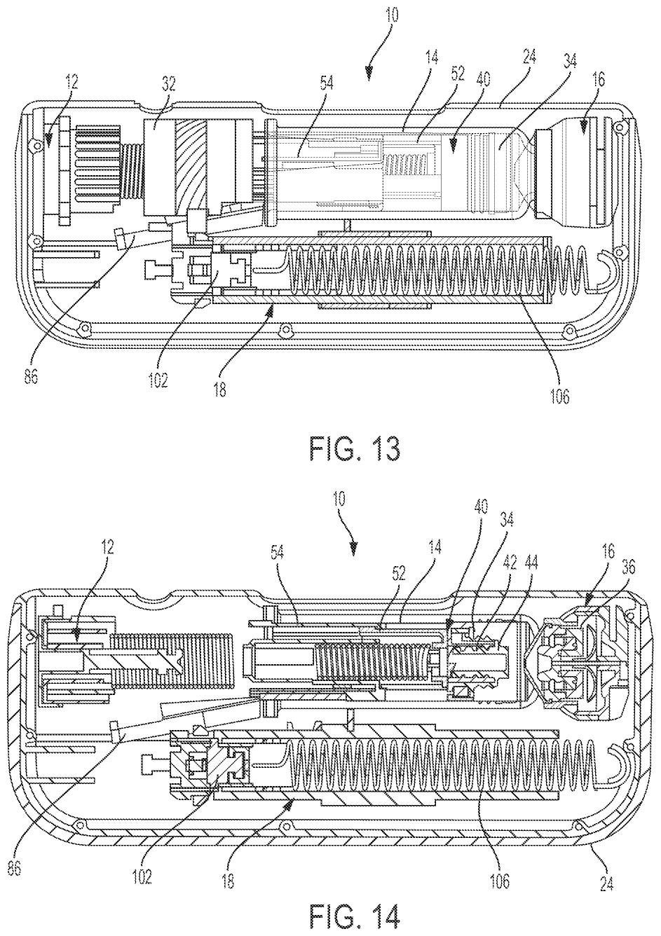

During the use position of the system 10, as shown in FIGS. 10-12, the needle 28 is in the extended position at least partially outside of the housing 20 with the drive assembly 12 moving the stopper 34 within the container 14 to deliver the medicament from the container 14, through the needle 28, and to the user. In the use position, the valve assembly 16 has already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the system 10, shown in FIGS. 13-15A, the needle 28 is in the retracted position and engaged with a pad 38 to seal the needle 28 and prevent any residual flow of fluid or medicament from the container 14. The container 14 and valve assembly 16 may be the container 14 and valve assembly 16 shown and described in International Publication No. WO 2015/081337, which is hereby incorporated by reference in its entirety.

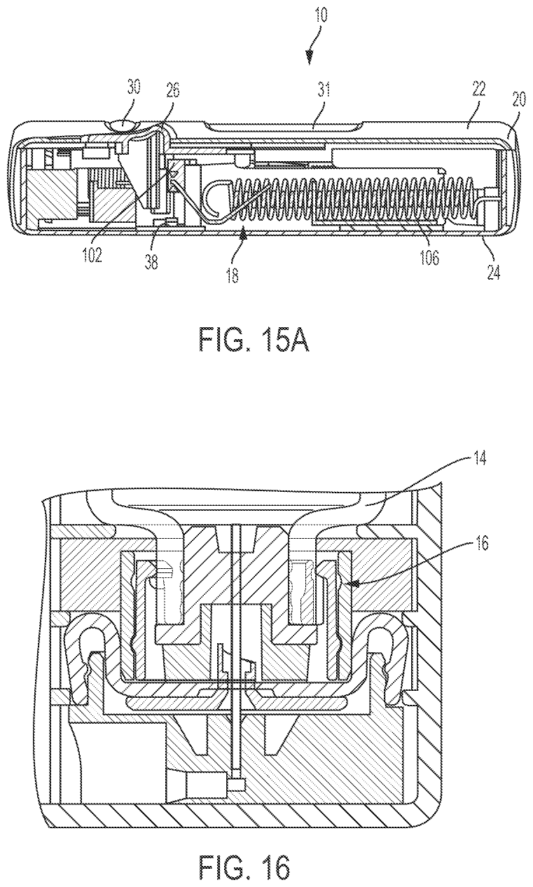

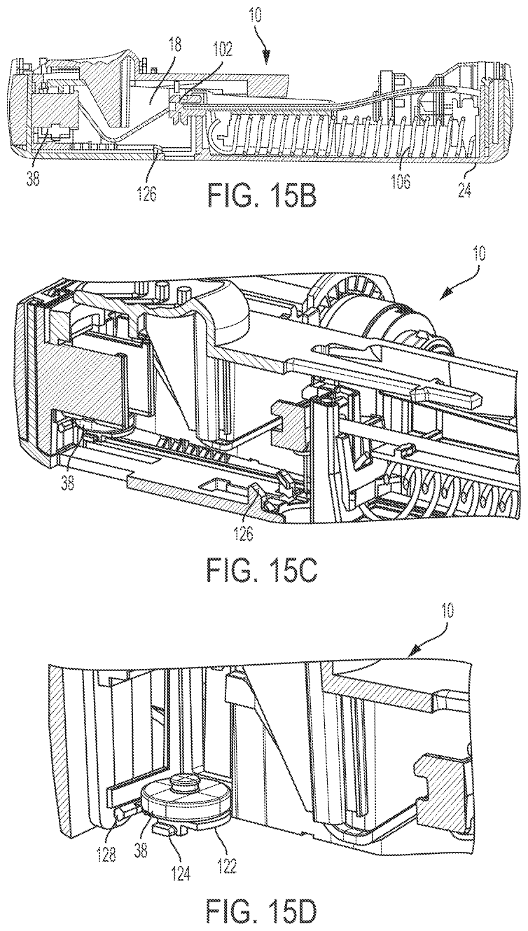

Referring to FIGS. 15A-15D, the pad 38 is biased into the pad as the needle actuator body 96 moves from the use position to the post-use position. In particular, the pad 38 is received by a pad arm 122 having a cam surface 124 that cooperates with a cam track 126 on the bottom portion 24 of the housing 20. The pad arm 122 is connected to the needle actuator body 96 via a torsion bar 128. The cam surface 124 is configured to engage the cam track 126 to deflect the pad arm 122 downwards thereby allowing the pad 38 to pass beneath the needle 28 before being biased upwards into the needle 28. The torsion bar 128 allows the pad arm 122 to twist about a pivot of the needle actuator body 96. The pad 38 may be press-fit into an opening of the pad arm 122, although other suitable arrangements for securing the pad 38 may be utilized.

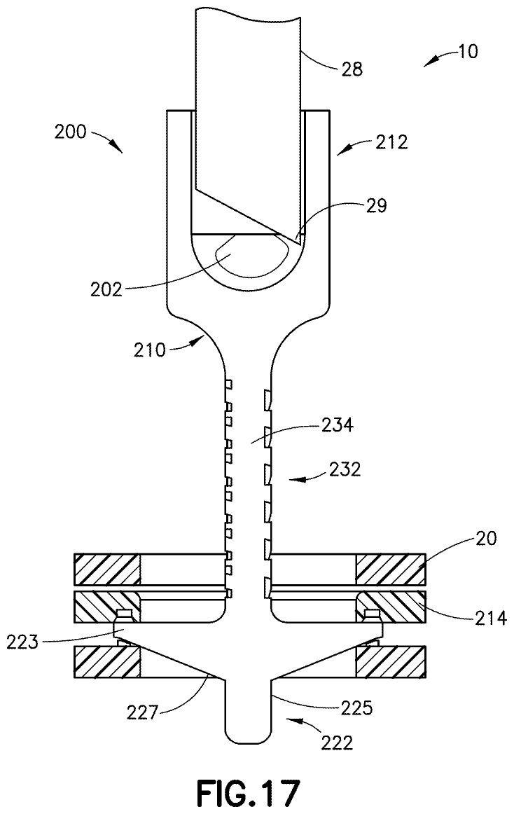

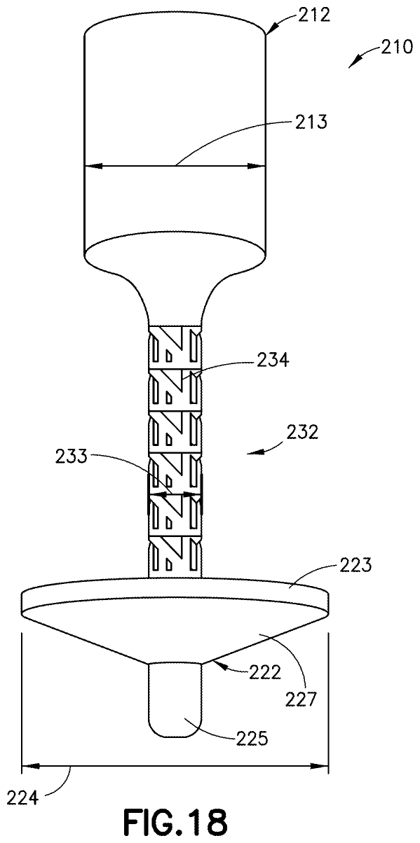

Referring to FIGS. 17 and 18, a barrier apparatus 200, in accordance with one non-limiting embodiment of the disclosed concept, includes a barrier 202 and a pull tab 210, each of which may be made of any suitable material in the art. The barrier 202 and the pull tab 210 can each be separate unitary components made from single pieces of material (e.g., injection molded pieces), or can together be one unitary component made from a single piece of material, as will be discussed below. In one example embodiment, the barrier 202 is an elastomeric sleeve for maintaining the sterility of the needle 28. In order to achieve this function, and as depicted in FIG. 17, the barrier 202 is configured to protectively surround at least a sharp 29 of the needle 28, which is configured to move into and out of the housing, as discussed above. It will be appreciated that prior to use, the barrier 202 is removed from the needle 28, e.g., via the pull tab 210, in order to allow the needle 28 to perform its intended function.

More specifically, the pull tab 210 includes a connecting portion 212, a gripping portion 222, and a body portion 232 extending between the connecting portion 212 and the gripping portion 222. The connecting portion 212 is connected to the barrier 202. In accordance with the disclosed concept, the connecting portion 212 is either directly connected with (e.g., wherein the pull tab 210 and the barrier 202 engage one another) or is integral with (e.g., wherein the pull tab 210 and the barrier 202 are formed as a unitary component (e.g., an injection molded piece) from one single piece of material) the barrier 202. As will be discussed below, this is advantageous, as compared with known barrier and pull tab arrangements (not shown), in which pull tabs are indirectly (e.g., via one or more intermediate parts or components) connected with barriers. The barrier 202 may be substantially enclosed by the connecting portion 212. In one example embodiment, the connecting portion 212 is connected to the barrier 202 by a press-fit mechanism. Thus, barrier 202 may be configured to be disposed within and engaged with a cavity of the connecting portion 212.

Referring to FIGS. 17 and 18, the gripping portion 222 is located apart from the connecting portion 212. The gripping portion 222 is also, as shown in FIG. 17, located at least partially external with respect to the housing 20 of the drug delivery system 10. As shown in FIG. 17, the gripping portion 222 is attached to a removal component 214 positioned at least partially external to the housing 20. The removal component 214 is configured to be grasped and pulled away from the housing 20 to removal the barrier apparatus 200 from the system 10 and expose the sharp 29 of the needle 28 just prior to using the system 10. Although the gripping portion 222 is attached to the removal component 214, the gripping portion 222 may be directly grasped to remove the barrier apparatus 200 or the gripping portion 222 may be formed integrally with the removal component 214. Referring again to FIG. 18, the gripping portion 222 has a base 223, a protrusion 225, and a frusto-conical shaped portion 227 extending between the base 223 and the protrusion 225. While a frusto-conical geometry is employed in the embodiment of FIGS. 17 and 18 to transition between the relatively wide base 223 and the relatively narrow protrusion 225, it will be appreciated that suitable alternative geometries are contemplated herein, without departing from the scope of the disclosed concept. During assembly of the barrier apparatus 200 to the system 10, in accordance with one non-limiting embodiment, the barrier 202 of the barrier apparatus 200 is first connected to the system 10 such that the barrier 202 encloses the sharp 29 of the needle 28 with the protrusion 225 configured to be grasped and pulled for threading the gripping portion 222 through the housing 20 and the removal component 214 such that the gripping portion 222 is engaged and secured to the removal component 214. The protrusion 225 may be subsequently removed after assembly. As shown in FIG. 17, the gripping portion 222 is pressed or confined between two portions of the removal component 214 to attach the gripping portion 222 to the removal component 214, although other suitable attachment arrangements may be utilized.

Continuing to refer to the example of FIGS. 17 and 18, it will be appreciated that the geometry of the pull tab 210 advantageously assists with maintaining the pull tab 210 in the housing 20 of the drug delivery system 10 prior to use while remaining connected to the barrier 202. More specifically, as shown in FIG. 18, the base 223, the connecting portion 212, and the body portion 232 have first, second, and third respective diameters 224,213,233. The second diameter 213 is less than the first diameter 224, and the third diameter 233 is less than the first and second diameters 224,213. As shown in FIG. 17, the relatively wide base 223 is maintained in the removal component 214.

In accordance with one non-limiting embodiment of the disclosed concept, the body portion 232 has a flexible portion 234 for reducing strain on the barrier 202. In this manner, undesirable movements of the barrier 202 are advantageously able to be minimized. As a result, undesirable movements of the needle 28 are likewise able to be significantly minimized, thereby maintaining its integrity for subsequent use by a user. Moreover, known pull tabs (not shown) for barriers generally have arms that close around the barrier upon removal, which rely on complex mechanical interactions and friction, and generally indirectly connect pull tabs with barriers that may not allow reliable removal of the barrier. In accordance with the disclosed concept, such mechanical interactions are simplified via the direct or integral connection between the connecting portion 212 and the barrier 202, thereby providing a more reliable removal of the barrier 202 upon disconnection of the barrier apparatus 200 from the system 10.

The flexible portion 234 is preferably more flexible than the connecting portion 212 and the gripping portion 222. More specifically, responsive to the same magnitude force being applied to the flexible portion 234, the connecting portion 212, and the gripping portion 222, and at angles perpendicular to a longitudinal axis of the pull tab, the flexible portion 234 is configured to deflect a greater distance from its original position than both the connecting and gripping portions 212,222. Moreover, in one example embodiment the flexible portion 234 extends from at or about the connecting portion 212 to at or about the gripping portion 222 (e.g., comprises substantially the entire body portion 232). Additionally, the body portion 232 in one example embodiment is elongated to allow for deep placement of the barrier 202, thus further minimizing disturbances of the barrier 202.

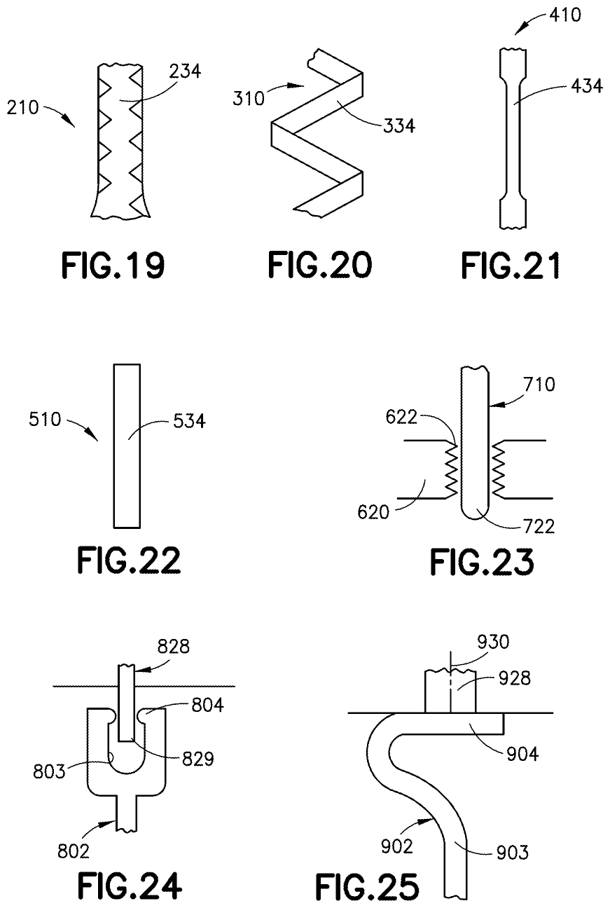

Referring to FIGS. 17-19, the flexible portion 234 is in the form of a webbed portion. In particular, the flexible portion 234 may include surface indentations or other structure to increase the flexibility of the flexible portion 234. However, other suitable configurations are contemplated by the disclosed concept. For example, in further non-limiting embodiments, FIGS. 20-22 show flexible portions 334,434,534 of pull tabs 310,410,510 in the form of a coiled portion, a segmented tube (e.g., having a varied diameter along a central axis), and a relatively stretchable straight segment, respectively. Accordingly, it will be appreciated that the strain relief in accordance with the disclosed concept can take many forms, provided the flexible portion reduces strain or movement of the barrier 202.

Referring to FIG. 23, rather than pressing the gripping portion 222 between portions of the removal component 214 as shown in FIG. 17, a portion 710 of the barrier apparatus 200 is compressed between serrated edges 622 of a removal component 620 with a protrusion 722 extending from the removal component 620 and operating in the same manner as the protrusion 225 discussed above and shown in FIG. 17.

Referring to FIG. 24, a barrier 802 according to an alternative non-limiting embodiment has a cylindrical-shaped surface 803 and an internal ledge 804 extending radially inwardly from the cylindrical-shaped surface 803. Prior to use, a needle 828 extends through the internal ledge 804 and the internal ledge 804 engages the needle 828. Furthermore, as shown, it is contemplated that a sharp 829 of the needle 828 need not be substantially engaged on all sides with the barrier 802.

Referring to FIG. 25, a barrier 902 according to an alternative non-limiting embodiment has an elongated portion 903 and a generally planar portion 904 extending from the elongated portion 903 and being located generally perpendicular with respect to a longitudinal axis 930 of a needle 928.

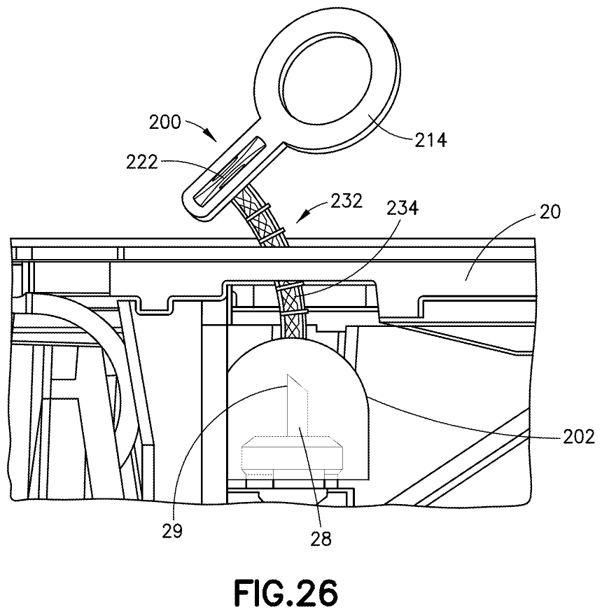

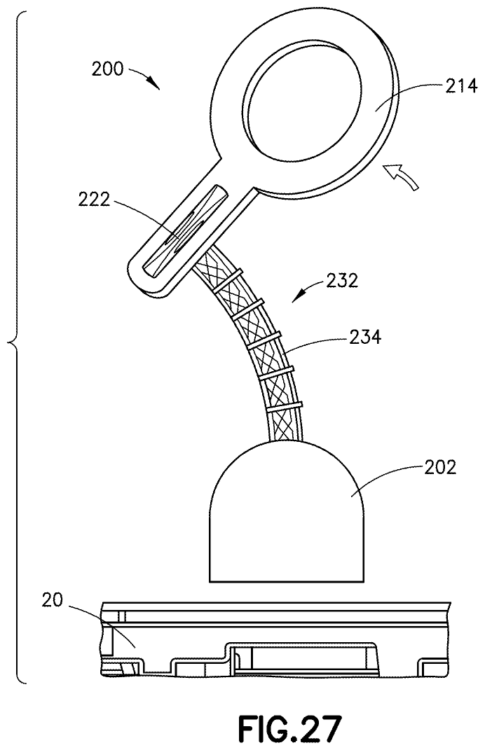

Referring to FIGS. 26 and 27, the flexible portion 234 of the barrier apparatus 200 prevents motion of the barrier 202 when the gripping portion 222 and the removal component 214 are moved laterally, which can compromise the sterility of the needle 28 and also cause undesirable contact between the needle 28 and the barrier 202. The flexible portion 234 of the barrier apparatus 200 is also configured to allow omnidirectional removal of the barrier apparatus 200 from the system 10 while providing reliable removal of the barrier 202 and without comprising the integrity of the system 10.

Elements of one disclosed aspect can be combined with elements of one or more other disclosed aspects to form different combinations, all of which are considered to be within the scope of the present concept.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A drug delivery system for injecting a medicament, the drug delivery system comprising:
a housing;
a needle having a sharp, the sharp being configured to move into and out of the housing; and
a barrier apparatus comprising:
a barrier configured to protectively surround at least the sharp of the needle, and
a pull tab comprising:
a connecting portion connected to the barrier,
a gripping portion disposed apart from the connecting portion and at least partially external with respect to the housing, and
a body portion extending between the connecting portion and the gripping portion,
wherein the body portion has a flexible portion for reducing strain on the barrier.

2. The drug delivery system according to claim 1, wherein the flexible portion is more flexible than the connecting portion and the gripping portion.

3. The drug delivery system according to claim 2, wherein the flexible portion extends from at or about the connecting portion to at or about the gripping portion.

4. The drug delivery system according to claim 3, wherein the flexible portion is a segmented tube.

5. The drug delivery system according to claim 3, wherein the flexible portion is a webbed portion.

6. The drug delivery system according to claim 3, wherein the flexible portion is a coiled portion.

7. The drug delivery system according to claim 3, wherein the flexible portion is a stretchable straight segment.

8. The drug delivery system according to claim 1, wherein the gripping portion has a base having a first diameter; and wherein the connecting portion has a second diameter less than the first diameter.

9. The drug delivery system according to claim 8, wherein the gripping portion further comprises a protrusion and a frusto-conical shaped portion extending between the base and the protrusion.

10. The drug delivery system according to claim 8, wherein the body portion has a third diameter less than the first diameter and the second diameter.

11. The drug delivery system according to claim 1, further comprising a removal component, the gripping portion attached to the removal component.

12. The drug delivery system according to claim 1, wherein the barrier comprises a cylindrical-shaped surface and an internal ledge extending radially inwardly from the cylindrical-shaped surface; wherein the needle extends through the internal ledge; and wherein the internal ledge engages the needle.

13. The drug delivery system according to claim 1, wherein the barrier comprises an elongated portion and a generally planar portion extending from the elongated portion of the barrier and being disposed generally perpendicular with respect to a longitudinal axis of the needle.

14. The drug delivery system according to claim 1, further comprising a removal component having a serrated portion, wherein the gripping portion is attached to the removal component by the serrated portion.

15. The drug delivery system according to claim 1, wherein the barrier comprises an elastomeric sleeve for maintaining the sterility of the needle.

16. A barrier apparatus for a drug delivery system, the drug delivery system comprising a housing and a needle having a sharp, the sharp being configured to move into and out of the housing, the barrier apparatus comprising:
a barrier configured to protectively surround at least the sharp of the needle; and
a pull tab comprising:
a connecting portion connected to the barrier,
a gripping portion disposed apart from the connecting portion and configured to be disposed at least partially external with respect to the housing, and
a body portion extending between the connecting portion and the gripping portion,
wherein the body portion has a flexible portion for reducing strain on the barrier.

17. The barrier apparatus according to claim 16, wherein the barrier and the pull tab are a unitary component made from one single piece of material.

18. The barrier apparatus according to claim 16, wherein the connecting portion is connected to the barrier by a press-fit mechanism.

19. The barrier apparatus according to claim 16, wherein the barrier is substantially enclosed by the connecting portion of the pull tab.

20. A pull tab for a barrier apparatus of a drug delivery system, the barrier apparatus comprising a barrier, the pull tab comprising:
a connecting portion configured to be connected to the barrier;
a gripping portion disposed apart from the connecting portion; and
a body portion extending between the connecting portion and the gripping portion,
wherein the body portion has a flexible portion for reducing strain on the barrier.
